(19) United States
(12) Patent Application Publication
(10) Pub. No.: US 2011/0053913 A1
(43) Pub. Date: Mar. 3, 2011

(54) Methods and Therapies for Alleviating Pain

(76) Inventors: Khem Jhamandas, Kingston (CA); Brian Milne, Kingston (CA); Catherine M. Cahill, Kingston (CA)

(21) Appl. No.: 12/823,934

(22) Filed: Jun. 25, 2010

Related U.S. Application Data
(60) Provisional application No. 61/220,282, filed on Jun. 25, 2009.

(51) Int. Cl. A61K 31/55; A61K 31/4164; A61K 31/4375; A61K 31/4178; A61P 29/00

(52) U.S. Cl. 514/214.02; 514/396; 514/280; 514/397

(57) ABSTRACT

A method for alleviating or delaying onset of pain in a subject via administration of an ultra low dose of alpha-2 receptor antagonist which does not significantly block or inhibit alpha-2 receptor activity is provided. A non-selective alpha-2 receptor antagonist or a selective alpha-2A receptor antagonist may be administered.

### RELATED APPLICATION

This application claims the benefit of the filing date of U.S. Provisional Patent Application No. 61/220,282, filed on 25 Jun. 2009, the contents of which are incorporated herein by reference in their entirety.

### FIELD

This invention relates generally to the field of analgesia. More particularly, this invention relates to methods and therapies for alleviating pain by administering an ultra low dose of an alpha-2 receptor antagonist.

### BACKGROUND

Inhibition of adrenergic alpha-2 receptors by alpha-2 receptor antagonists such as atipemazole and yohimbine is known to elicit effects such as changes heart rate and blood pressure. It is also known that the actions of alpha-2 receptor agonists are blocked by alpha-2 receptor antagonists such as atipemazole and yohimbine.

WO 03/099289 discloses a method for alleviating pain in a subject by administering a composition containing an alpha-adrenergic agonist and a selective alpha-2A antagonist. Our previous work has shown that a combination therapy comprising an opioid analgesic agent and an alpha-2 receptor antagonist provides effective analgesia while avoiding negative side effects of opioids such as tolerance and addiction (see, e.g., U.S. patent application Ser. No. 11/515,301).

More desirable would be a therapy for pain management that does not employ opioid agents. However, little work has been done in this area. High doses of atipemazole were evaluated in pain models by Kauppila et al. (Pharmacology Biochemistry and Behavior 1998 59:477-485), but the effects were attributed to impaired motor function.

### SUMMARY

Also described herein is method for alleviating or delaying onset of pain in a subject comprising administering to the subject an ultra low dose of an alpha-2 receptor antagonist which does not significantly reduce, block, or inhibit alpha-2 receptor activity.

Described herein is a method for alleviating or delaying onset of pain in a subject, comprising systemically administering to the subject an ultra low dose of an alpha-2 receptor antagonist which does not significantly reduce, block, or inhibit alpha-2 receptor activity.

In one embodiment, the alpha-2 receptor antagonist is administered subcutaneously. In another embodiment, the alpha-2 receptor antagonist is atipemazole. The atipemazole may be administered subcutaneously.

In other embodiments, the alpha-2 receptor antagonist may be selected from the group consisting of atipemazole (or atipamezol or atipamzole), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563, A 80246 mesylate, ARC 239 dihydrochloride, imiloxan hydrochloride, JP 1302 dihydrochloride, rauwolscine hydrochloride, RS 79948, spiroxatrine, and yohimbine.

The alpha-2 receptor antagonist may be selected from the group consisting of venlafaxine, doxazosin, phentolamine, dihydroergotamine, ergotamine, phenothiazines, phenoxybenzamine, piperoxane, prazosin, tamsulosin, terazosin, mianserin, and tolazoline.

The alpha-2 receptor antagonist may be a selective alpha-2A receptor antagonist. The selective alpha-2A receptor antagonist may be BRL 44408 or BRL 48962.

In one embodiment the selective alpha-2A receptor antagonist is BRL 44408. The BRL 44408 may be administered subcutaneously.

Also described herein is a method for alleviating or delaying onset of pain in a subject, comprising administering to the subject an ultra low dose of an alpha-2 receptor antagonist which does not significantly reduce, block, or inhibit alpha-2 receptor activity; wherein the alpha-2 receptor antagonist is BRL 44408.

In one embodiment the BRL 44408 is administered intrathecally.

Methods and therapies described herein are useful for management of pain including, but not limited to, acute and/or chronic post-surgical pain, obstetrical pain, acute and/or chronic inflammatory pain, pain associated with conditions such as multiple sclerosis and/or cancer, pain associated with trauma, pain associated with migraines, neuropathic pain, central pain and chronic pain syndrome of a non-malignant origin such as chronic back pain. Compositions of the present invention are also useful as cough suppressants, in reduction and/or prevention of diarrhea, in treatment of pulmonary edema and in alleviating physical dependence and/or addiction to opioid receptor agonists.

It is understood that such treatment may also be commenced prior to pain or suffering (i.e., prophylactically, when the subject is at risk for such suffering).

Yet a further aspect of each of the above methods is that the opioid receptor antagonist is administered or formulated in an amount which does not elicit a substantial undesirable side effect.

### BRIEF DESCRIPTION OF THE FIGURES

FIGS. 1A and 1B are line graphs showing the effects of the alpha-2 receptor antagonist atipemazole at inhibiting analgesia by the alpha-2 receptor agonist clonidine in a tail flick test (FIG. 1A) and a paw pressure test (FIG. 1B) in rats. Clonidine was administered intrathecally at 200 nmoles which is equal to 53.2 micrograms per rat. Rats were co-administered atipemazole intrathecally at 0 micrograms/rat (open circle), 1 microgram/rat (filled square), 5 micrograms/rat (filled triangle), and 10 micrograms/rat (inverted filled triangle).

FIGS. 2A and 2B are line graphs showing the effects of the alpha-2 receptor antagonist atipemazole administered at a dose ineffective at causing alpha-2 receptor blockade on acute tolerance to the analgesic actions of spinal morphine in the tail flick test (FIG. 2A) and paw pressure test (FIG. 2B) in rats. In this study, acute tolerance was produced by delivering three intrathecal successive injections (depicted by vertical arrows) of morphine (15 μg) at 90 minute intervals (depicted by open circles). A second group of rats received a combination of morphine (15 μg) and a fixed dose of atipemazole (0.8 ng) (depicted by filled circles). The effects of atipemazole alone (0.8 ng)(depicted as filled triangles) and normal saline (20 μl)(depicted as open squares) were also evaluated by injecting these at 90 minute intervals.

FIGS. 3A and 3B are line graphs showing the effects of administration of the alpha-2 receptor antagonist atipemazole, at doses ineffective at causing alpha-2 receptor blockade, on the acute morphine analgesia in the tail flick (FIG. 3A) and paw pressure test (FIG. 3B) in rats. Rats administered morphine (15 μg) alone are depicted by open circles. Rats administered morphine (15 μg) and atipemazole at 0.8 ng are depicted by filled triangles. Rats administered morphine (15 μg) and atipemazole at 0.08 ng are depicted by inverted filled triangles. Rats administered atipemazole alone at 0.8 ng are depicted by open triangles.

FIGS. 4A and 4B are line graphs showing the antagonistic effects of the alpha-2 receptor antagonist yohimbine at inhibiting spinal analgesia by the alpha-2 receptor agonist clonidine in the tail flick test (FIG. 4A) and paw pressure test (FIG. 4B) in rats. Rats were administered clonidine (13.3 μg) intrathecally alone (open circles), yohimbine (30 μg) intrathecally alone (open triangles), or clonidine (13.3 μg) and yohimbine (30 μg) intrathecally (filled squares).

FIGS. 5A and 5B are line graphs showing the antagonistic effects of the alpha-2 receptor antagonist yohimbine at inhibiting spinal morphine analgesia in the tail flick test (FIG. 5A) and paw pressure test (FIG. 5B). Rats were administered morphine (15 μg) intrathecally alone (open circles), yohimbine (30 μg) intrathecally alone (open triangles), or morphine (15 μg) and yohimbine (30 μg) intrathecally (filled squares).

FIGS. 6A and 6B are line graphs showing the effects of the alpha-2 receptor antagonist yohimbine administered at a dose ineffective at causing alpha-2 receptor blockade on acute tolerance to the analgesic actions of spinal morphine in the tail flick test (FIG. 6A) and paw pressure test (FIG. 6B) in rats. In this study, acute tolerance was produced by delivering three intrathecal successive injections (indicated by arrowheads) of morphine (15 μg) at 90 minute intervals (depicted by open circles). Other groups of rats received a combination of morphine (15 μg) and a fixed dose of yohimbine of 0.0048 ng (depicted by filled squares), 0.024 ng (filled triangles), or 0.24 ng (inverted filled triangles). The effects of yohimbine alone (0.024 ng; depicted as open triangles) and normal saline (20 μl; depicted as Xs) were also evaluated by injecting these at 90 minute intervals.

FIGS. 7A and 7B are line graphs showing the antagonistic effects of the alpha-2 receptor antagonist mirtazapine at inhibiting spinal analgesia by the alpha-2 receptor agonist clonidine in the tail flick test (FIG. 7A) and paw pressure test (FIG. 7B) in rats. Rats were administered clonidine (13.3 μg) intrathecally alone (open squares) or clonidine (13.3 μg) and mirtazapine (2 μg) intrathecally (filled squares).

FIGS. 8A and 8B are line graphs showing the effects of the alpha-2 receptor antagonist mirtazapine administered at a dose ineffective at causing alpha-2 receptor blockade on acute tolerance to the analgesic actions of spinal morphine in the tail flick test (FIG. 8A) and paw pressure test (FIG. 8B) in rats. In this study, acute tolerance was produced by delivering three intrathecal successive injections (indicated by arrowheads) of morphine (15 μg) at 90 minute intervals (depicted by open circles). Another group of rats received a combination of morphine (15 μg) and a fixed dose of mirtazapine of 0.02 ng (depicted by filled triangles). The effects of normal saline (20 μl; depicted as Xs) injected at 90 minute intervals were also evaluated.

FIGS. 9A and 9B are line graphs showing the antagonistic effects of the alpha-2 receptor antagonist idazoxan at inhibiting spinal analgesia by the alpha-2 receptor agonist clonidine in the tail flick test (FIG. 9A) and paw pressure test (FIG. 9B) in rats. Rats were administered clonidine (13.3 μg) intrathecally alone (open squares), idazoxan (10 μg intrathecally alone (open diamonds), clonidine (13.3 μg) and idazoxan (10 μg) intrathecally (filled squares), or saline (20 μl; depicted by Xs).

FIGS. 10A and 10B are line graphs showing the effects of the alpha-2 receptor antagonist idazoxan administered at a dose ineffective at causing alpha-2 receptor blockade on acute tolerance to the analgesic actions of spinal morphine in the tail flick test (FIG. 10A) and paw pressure test (FIG. 10B) in rats. In this study, acute tolerance was produced by delivering three intrathecal successive injections of morphine (15 μg) at 90 minute intervals (depicted by open circles). Other groups of rats received idazoxan alone at 0.016 ng (depicted by open triangles) or 0.08 ng (depicted by inverted open triangles), or a combination of morphine (15 μg) and a fixed dose of idazoxan of 0.008 ng (depicted by inverted filled triangles), 0.016 ng (depicted by filled triangles) or 0.08 ng (depicted by filled diamonds). The effects of normal saline (20 μl; depicted as Xs) injected at 90 minute intervals were also evaluated.

FIGS. 11A and 11B are line graphs showing the effects of the alpha-2 receptor antagonist atipemazole on acute tolerance to the analgesic actions of systemic morphine in the tail flick test (FIG. 11A) and paw pressure test (FIG. 11B) in rats (n=4 rats per group). FIGS. 11A and 11B also show the analgesic effect of atipemazole administered alone. In this study, acute tolerance was produced by delivering three subcutaneous (sc) successive injections (depicted by vertical arrows) of morphine (2.5 mg/kg sc) at 90 minute intervals (depicted by open circles). A second group of rats received a combination of morphine (2.5 mg/kg sc) and a dose of atipemazole of 20 ng/kg sc (depicted by filled triangles). A third group of rats received a combination of morphine (2.5 mg/kg sc) and a dose of atipemazole of 200 ng/kg sc (depicted by filled inverted triangles). The effects of atipemazole alone at 20 ng/kg sc (depicted by open triangles) and 200 ng/kg sc (depicted by open inverted triangles) and normal saline (1 ml/kg) (depicted as open squares) were also evaluated by injecting these subcutaneously at 90 minute intervals. * designates p<0.05, ** designates p<0.01, and *** designates p<0.001 compared to corresponding effect of morphine (2.5 mg/kg sc) alone.

FIG. 12 is a line graph showing the antagonistic effect of the alpha-2A receptor antagonist BRL 44408 at inhibiting spinal analgesia by the alpha-2 receptor agonist clonidine in the tail flick test in rats. Rats were administered clonidine (13.3 μg) intrathecally alone (closed squares), clonidine (13.3 μg) and BRL 44408 (3.3 μg) intrathecally (inverted triangles), or clonidine (13.3 μg) and BRL 44408 (16.5 μg) intrathecally (triangles).

FIGS. 13A and 13B are line graphs showing the effects of the alpha-2A receptor antagonist BRL44408 in augmenting the analgesic action of a single administration of morphine in the tail flick test (FIG. 13A) and paw pressure test (FIG. 13B) in rats. FIGS. 13A and 13B also show the analgesic effect of BRL 44408 administered alone. BRL 44408 was administered at a dose ineffective at causing alpha-2A receptor blockade. Morphine was administered intrathecally alone (15 μg) (circles) or with BRL 44408 (1.65 ng) (filled triangles). BRL 44408 was administered intrathecally alone (1.65 ng) (open triangles).

FIGS. 14A and 14B are line graphs showing the effects of the alpha-2A receptor antagonist BRL 44408, on acute tolerance to the analgesic actions of spinal morphine in the tail flick test (FIG. 14A) and paw pressure test (FIG. 14B) in rats. FIGS. 14A and 14B also show the analgesic effect of BRL 44408 administered alone. BRL 44408 was administered at a dose ineffective at causing alpha-2A receptor blockade. In this study, acute tolerance to morphine was produced by delivering three intrathecal successive injections (depicted by vertical arrows) of morphine (15 μg) at 90 minute intervals (depicted by open circles). A second group of rats received a combination of morphine (15 μg) and a dose of BRL 44408 of 1.65 ng (depicted by filled triangles). The effects of BRL 44408 alone at 1.65 ng (depicted by open triangles) and normal saline (20 μl) (depicted as open squares) were also evaluated by injecting these intrathecally at 90 minute intervals.

FIGS. 15A and 15B are line graphs showing analgesia produced by the alpha-2A receptor antagonist atipemazole in the formalin test in rats. Atipemazole or vehicle was injected by subcutaneous injection 20 minutes prior to formalin injection into the plantar surface of the rat hind paw. The data show that atipemazole (alone) was as effective as morphine. Data represent means+/−s.e.m. for N=4.

### DETAILED DESCRIPTION OF EMBODIMENTS

Described herein are methods and therapies for alleviating and/or managing pain, comprising administering an ultra-low dose of an alpha-2 receptor antagonist. The methods and therapies described herein are useful in various applications including but not limited to: pain management, e.g. management of acute or chronic post-surgical pain, obstetrical pain, acute or chronic inflammatory pain, pain associated with conditions such as multiple sclerosis or cancer, pain associated with trauma, pain associated with migraines, neuropathic pain, and central pain; management of chronic pain syndrome of a non-malignant origin such as chronic back pain; cough suppression; reducing and/or preventing diarrhea; and treating pulmonary edema. In particular, it is expected that the methods and therapies described herein may be useful as a substitute for opioid therapy, thereby avoiding potential problems associated with opioids such as addiction and tolerance.

An alpha-2 receptor antagonist useful in the methods described herein may be any compound that partially or completely reduces, inhibits, blocks, inactivates and/or antagonizes the binding of an alpha-2 receptor agonist to its receptor to any degree and/or the activation of an alpha-2 receptor to any degree. Thus, the term alpha-2 receptor antagonist is also meant to include compounds that antagonize the agonist in a competitive, irreversible, pseudo-irreversible and/or allosteric mechanism.

Examples of alpha-2 receptor antagonists useful in the therapies and methods described herein include, but are in no way limited to atipemazole (or atipamezol or atipamezole), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563, BRL 44408, yohimbine, A 80246 mesylate, ARC 239 dihydrochloride, imiloxan hydrochloride, JP 1302 dihydrochloride, prasozin hydrochloride, rauwolscine hydrochloride, RS 79948, and spiroxatrine. Additional examples of agents which exhibit some alpha 2 and/or alpha 1 receptor antagonistic activity and thus may be useful in the present invention include, but are not limited to, venlafaxine, doxazosin, phentolamine, dihydroergotamine, ergotamine, phenothiazines, phenoxybenzamine, piperoxane, prazosin, tamsulosin, terazosin, mianserin, and tolazoline. In one embodiment, the alpha-2 receptor antagonist is a non-selective alpha-2 receptor subtype. In another embodiment, the alpha-2 receptor antagonist is selective for alpha-2A receptors. Examples of selective alpha-2A receptor antagonists include, but are not limited to, BRL 44408 (2-[2H-(1-Methyl-1,3-dihydroisoindole)methyl]-4,5-dihydroimidazole maleate; Sigma-Aldrich, St. Louis. MO) and BRL 48962(R-(−)-2,3-dihydroisoindolymethylimidazoline), the R-enantiomer of BRL 44408 (Beeley et al. Biorganic and Medicinal Chemistry 1995 3:1693-1698). In the methods described herein, the alpha-2 receptor antagonist is administered at an ultra-low dose.

Compositions as well as methods described herein may comprise an ultra-low dose of more than one alpha-2 receptor antagonist.

As used herein, the term “ultra-low dose” refers to an amount of alpha-2 receptor antagonist lower than that established by those skilled in the art to significantly reduce, block, or inhibit alpha-2 receptor activity.

As used herein, the term “amount” is intended to refer to the quantity of alpha-2 receptor antagonist administered to a subject. The term “amount” encompasses the term “dose” or “dosage”, which is intended to refer to the quantity of alpha-2 receptor antagonist administered to a subject at one time or in a physically discrete unit, such as, for example, in a pill, injection, or patch (e.g., transdermal patch). The term “amount” also encompasses the quantity of alpha-2 receptor antagonist administered to a subject, expressed as the number of molecules, moles, grams, or volume per unit body mass of the subject, such as, for example, mol/kg, mg/kg, ng/kg, ml/kg, or the like, sometimes referred to as concentration administered.

In accordance with embodiments described herein, administration to a subject of a given amount of alpha-2 receptor antagonist results in an effective concentration of the antagonist in the subject's body. As used herein, the term “effective concentration” is intended to refer to the concentration of alpha-2 receptor antagonist in the subject's body (e.g., in the blood, plasma, or serum, at the target tissue(s), or site(s) of action) capable of producing a desired therapeutic effect. The effective concentration of alpha-2 receptor antagonist in the subject's body may vary among subjects and may fluctuate within a subject over time, depending on factors such as, but not limited to, the condition being treated, genetic profile, metabolic rate, biotransformation capacity, frequency of administration, formulation administered, elimination rate, and rate and/or degree of absorption from the route/site of administration. For at least these reasons, for the purpose of this disclosure, administration of alpha-2 receptor antagonist is conveniently provided as amount or dose of alpha-2 receptor antagonist. The amounts, dosages, and dose ratios provided herein are exemplary and may be adjusted, using routine procedures such as dose titration, to provide an effective concentration.

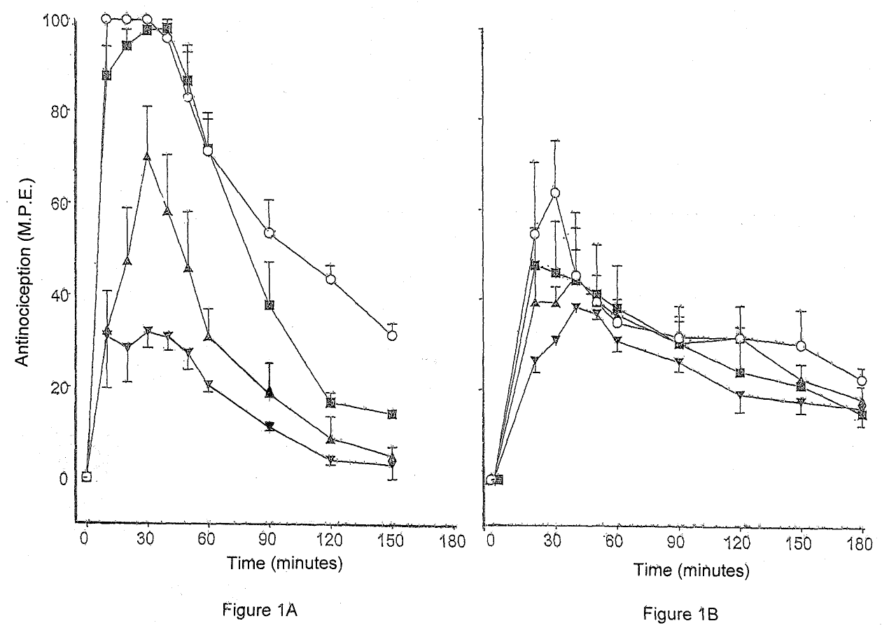

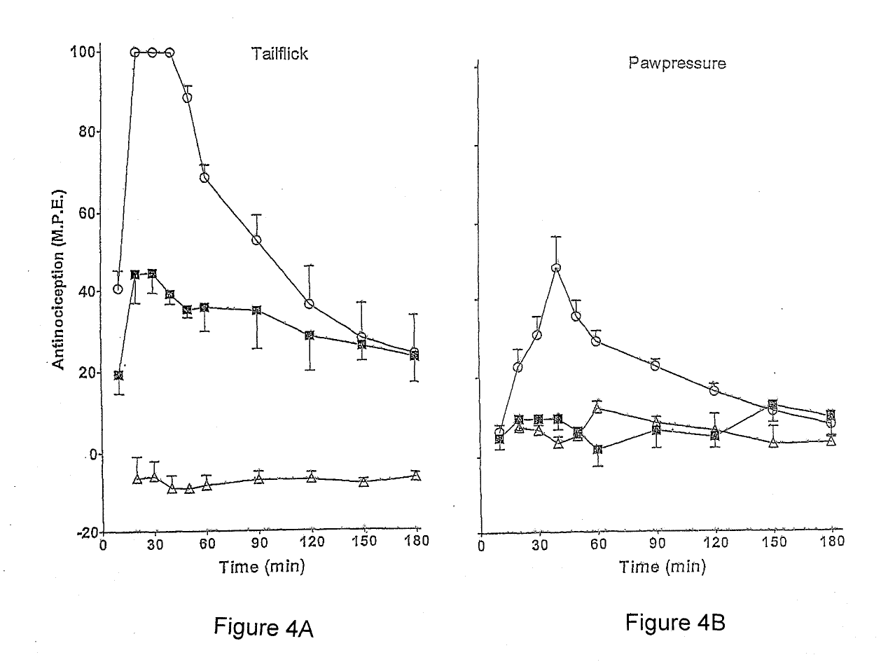

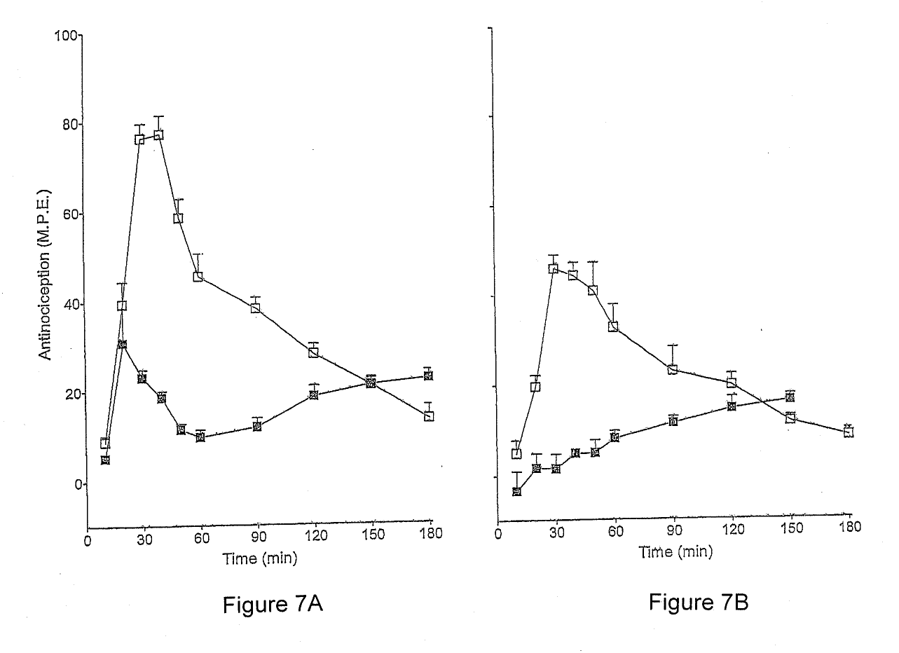

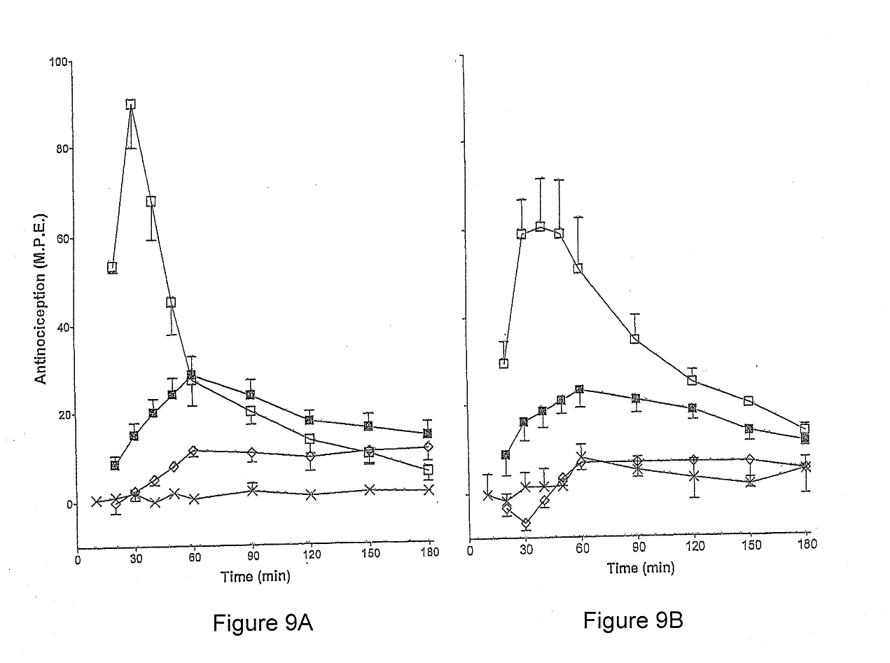

For example, in one embodiment, an ultra-low dose of alpha-2 receptor antagonist is an amount ineffective at alpha-2 receptor blockade as measured in experiments such as set forth in FIGS. 1A and 1B, FIGS. 4A and 4B, FIGS. 7A and 7B and FIGS. 9A and 9B. As will be understood by the skilled artisan upon reading this disclosure, however, other means for measuring alpha-2 receptor antagonism can be used. Based upon these experiments, ultra-low doses of atipemazole which potentiate the analgesic action of the opioid morphine were identified as being 12,000-fold to 120,000-fold lower than the dose producing a blockade of the spinal alpha-2 receptors, as evidenced by antagonism of intrathecal clonidine (alpha-2 agonist) analgesia (FIGS. 1A and 1B). Ultra-low doses of yohimbine which potentiate the analgesic action of the opioid morphine were identified as being 6,000 to 6,250,000-fold lower than the dose producing a blockade of the spinal alpha-2 receptors, as evidenced by antagonism of intrathecal clonidine (alpha-2 agonist) analgesia (FIGS. 4A and 4B). Ultra-low doses of mirtazapine which potentiate the analgesic action of the opioid morphine were identified as 10,000 to 100.000-fold lower than the dose producing a blockade of the spinal alpha-2 receptors, as evidenced by antagonism of intrathecal clonidine (alpha-2 agonist) analgesia (FIGS. 7A and 7B). Ultra-low doses of idazoxan which potentiate the analgesic action of the opioid morphine were identified as 125,000 to 1,250,000-fold lower than the dose producing a blockade of the spinal alpha-2 receptors, as evidenced by antagonism of intrathecal clonidine (alpha-2 agonist) analgesia (FIGS. 9A and 9B). Ultra-low doses useful in the present invention for other alpha-2 receptor antagonists as well as other therapeutic actions of opioids can be determined routinely by those skilled in the art in accordance with the known effective concentrations as alpha-2 receptor blockers and the methodologies described herein for atipemazole, yohimbine, mirtazepine and/or idazoxan. In general, however, the term “ultra-low” refers to a dose at least 1,000- to 6,250,000-fold lower than a dose producing at least partial blockade of alpha-2 receptors.

Another exemplary embodiment of an “ultra-low” dose is an amount of alpha-2 receptor antagonist that does not elicit a substantial undesirable side effect.

The term “substantial undesirable side effect” as used herein refers to a response in a subject to the alpha-2 receptor antagonist which cannot be controlled in the subject and/or endured by the subject and/or could result in discontinued treatment of the subject with the alpha-2 receptor antagonist.

Examples of such side effects include, but are not limited to, sedation, euphoria, dysphoria, memory impairment, hallucination, depression, headache, hyperalgesia, constipation, insomnia, body aches and pains, change in libido, nausea and vomiting, pruritus, dizziness, fainting (i.e., syncope), nervousness and/or anxiety, irritability, psychoses, tremors, changes in heart rhythm, decrease in blood pressure, elevated in blood pressure, elevated heart rate, risk of heart failure, temporary muscle paralysis and diarrhea.

The dose of alpha-2 receptor antagonist in the methodologies described herein is an amount that achieves an effective concentration and/or produces a desired therapeutic effect. For example, in a rat, an analgesic effect may be obtained using a systemic dosage of alpha-2 antagonist between about 10 ng/kg to about 500 ug/kg, or about 10 ng/kg to about 200 ug/kg, or about 10 ng/kg to about 10 ug/kg, or about 20 ng/kg to about 20 ug/kg, depending upon, but not limited to, the alpha-2 receptor antagonist selected, the route of administration, the frequency of administration, the formulation administered, and/or the condition being treated. For example, in a rat, an analgesic effect may be obtained by a dosage of alpha-2 antagonist between about 1 pg to about 5 ug, or about 1 pg to about 1 ug, or about 1 pg to about 100 ng, or about 10 pg to about 1 ug, delivered intrathecally.

For purposes of the present invention, the terms “therapeutic effect” or “therapeutic activity” or “therapeutic action” refer to a desired pharmacological activity of an alpha-2 receptor antagonist useful in pain management. In one embodiment, a therapeutic effect is associated with the inhibition, reduction, prevention or treatment of a condition that may be treated with an opioid receptor agonist. Examples include, but are not limited to, pain, coughs, diarrhea, pulmonary edema and addiction to opioid receptor agonists. That is, a therapeutic effect is meant to include a pharmacological activity measurable as an end result, i.e., alleviation of pain or cough suppression, as well as a pharmacological activity associated with a mechanism of action linked to the end desired result. In one embodiment, the “therapeutic effect” or “therapeutic activity” or “therapeutic action” is alleviation or management of pain.

The term “tolerance” as used herein refers to a loss of level of drug-induced response and drug potency. For example, tolerance is produced by many opioid receptor agonists, and particularly opioids. Chronic or acute tolerance can be a limiting factor in the clinical management of opioid drugs as opioid potency is decreased upon exposure to the opioid. The term “chronic tolerance” refers to a decrease in level of drug-induced response and drug potency which can develop after drug exposure over several or more days. The term “acute tolerance” refers to a loss in drug potency which can develop after drug exposure over several hours (Fairbanks and Wilcox J. Pharmacol. Exp. Therapeutics. 1997 282:1408-1417; Kissin et al. Anesthesiology 1991 74:166-171). Loss of opioid drug potency may also be seen in pain conditions such as neuropathic pain without prior opioid drug exposure as neurobiological mechanisms underlying the genesis of tolerance and neuropathic pain are similar (Mao et al. Pain 1995 61:353-364). This is also referred to as acute tolerance. Tolerance to opioids has been explained in terms of opioid receptor desensitization or internalization although exposure to morphine, unlike most other mu opioid receptor agonists, does not produce receptor internalization. It has also been explained on the basis of an adaptive increase in levels of pain transmitters such as glutamic substance P or CGRP. Inhibition of tolerance and maintenance of drug potency are important therapeutic goals in pain management which may be achieved by the therapies described herein. In particular, the methods and therapies described herein do not employ opioids and therefore avoid negative side effects such as tolerance.

One skilled in the art would know how to determine a suitable dose of alpha-2 receptor antagonist to achieve the desired therapeutic effect based upon the disclosure provided herein. For example, any alpha 2 receptor antagonist may be tested in animals using one or more available tests, including, but not limited to, tests for analgesia such as thermal, mechanical, and the like, or tests for neuropathic, inflammatory or nociceptive pain, or any other tests useful for assessing antinociception as well as other therapeutic actions of opioid receptor agonists. Non-limiting examples for testing analgesia typically used with rats include the thermal tail flick and mechanical paw pressure antinociception assays, and the formalin inflammatory pain model. To the extent that dosages determined in animal studies require adjustment or scaling for use in humans, such is a well-established and routine practice in the art. See, for example, Mordenti J. Pharmaceut. Sci. 1986 75:1028-1040. Furthermore, routine practices such as dose titration may be performed to determine a suitable dosage or dosage range.

The ability of exemplary alpha-2 receptor antagonist therapies to inhibit acute or chronic pain was demonstrated in tests of thermal (rat tail flick), mechanical (rat paw pressure), and inflammatory (formalin) antinociception. In these experiments, exemplary alpha-2 receptor antagonists included atipemazole and BRL 44408.

Previous studies investigated the ability of the alpha-2 receptor antagonists atipemazole, yohimbine, and idazoxan to augment opioid (e.g., morphine) analgesia and reduce tolerance to opioids. The drugs were administered by intrathecal injection. As a control, the alpha-2 receptor antagonists were also administered alone.

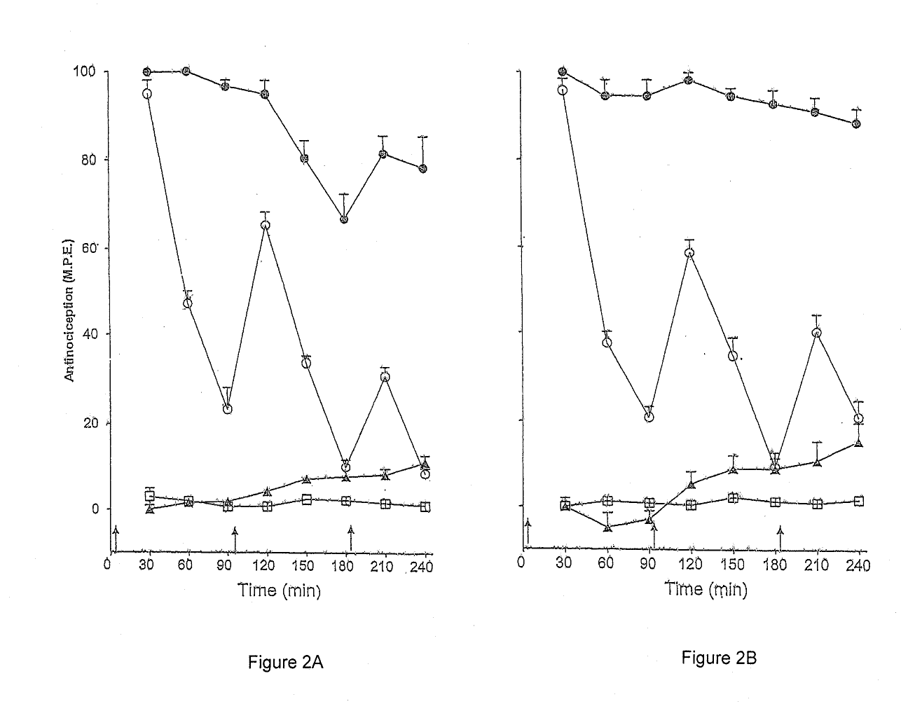

For example, FIGS. 2A and 2B illustrate effects of an ultra-low dose of atipemazole on the acute tolerance to the analgesic actions of spinal morphine. Administration of three successive doses of morphine (15 μg) at 90 minute intervals resulted in a rapid and progressive reduction of the analgesic response. At the end of the 240 minute test period, the analgesic effect of morphine observed after the first injection had declined by nearly 80%. However, administration of atipemazole (0.8 ng) with morphine prevented the decline of the analgesic effect of morphine. Indeed, the response to the combination remained near maximal value during the entire test period. The repeated administration of atipemazole alone produced an incremental but weak analgesic response. The three successive saline injections did not produce significant analgesic effect in either test.

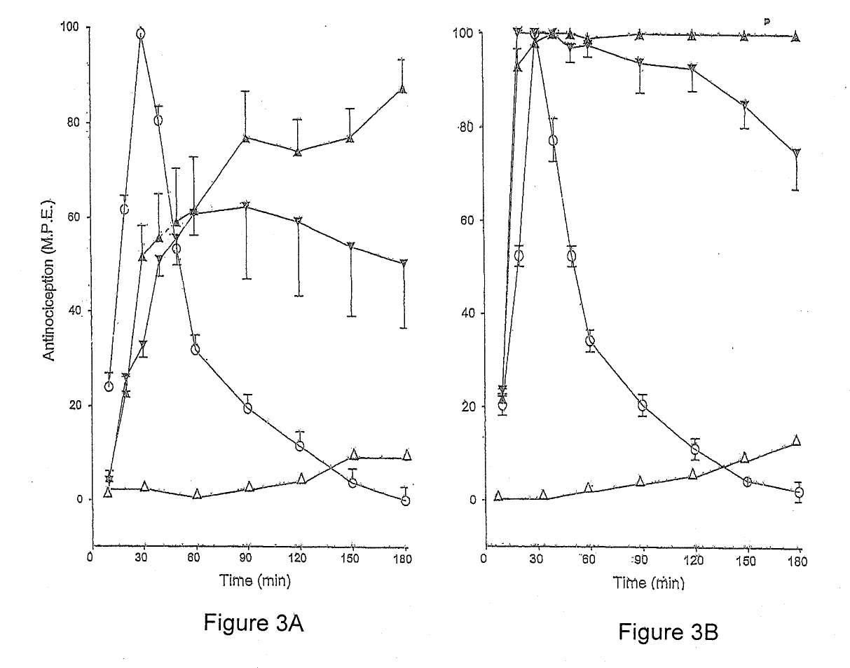

Similarly, FIGS. 3A and 3B show that atipemazole, when administered at an ultra-low dose of 0.8 or 0.08 ng, potentiates opioid analgesia, and that atipemazole administered alone has a weak, albeit insignificant, analgesic effect.

The analgesic effect of intrathecal administration of an ultra-low dose of atipemazole, when administered alone, depicted in FIGS. 2 and 3, may also be indicative of this therapy potentiating endogenous opioids such as endorphins (examples include beta-endorphins dynorphins and enkephalins) as well. Thus, described herein are methods for potentiating the therapeutic actions of an endogenous opioid in a subject (not being administered an exogenous opioid) upon intrathecal administration of an ultra-low dose alpha-2 receptor antagonist to the subject.

Intrathecal administration of ultra-low doses of alpha-2 receptor antagonists alone to alleviate or delay onset of pain may also be independent of potentiation of endogenous opioids.

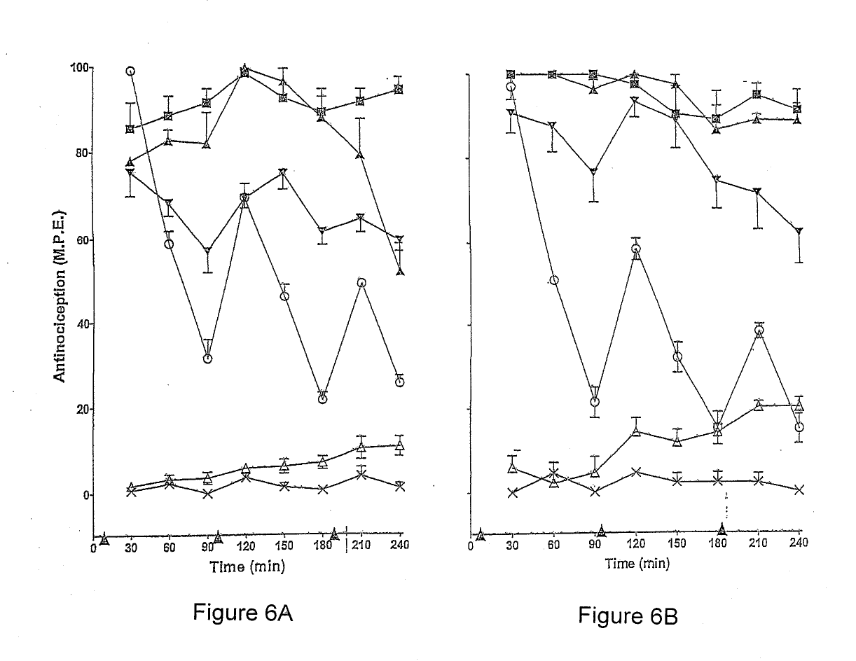

However, FIGS. 6A and 6B, which show the effects of intrathecal administration of an ultra-low dose of yohimbine on acute tolerance to the analgesic actions of spinal morphine, also show that repeated administration of yohimbine (0.024 ng) alone produced no significant analgesic response.

Similarly, FIGS. 9A and 9B, which show the effects of intrathecal administration of an ultra-low dose of idazoxan on acute tolerance to the analgesic actions of spinal morphine, also show that repeated administration of idazoxan alone produced no significant analgesic response.

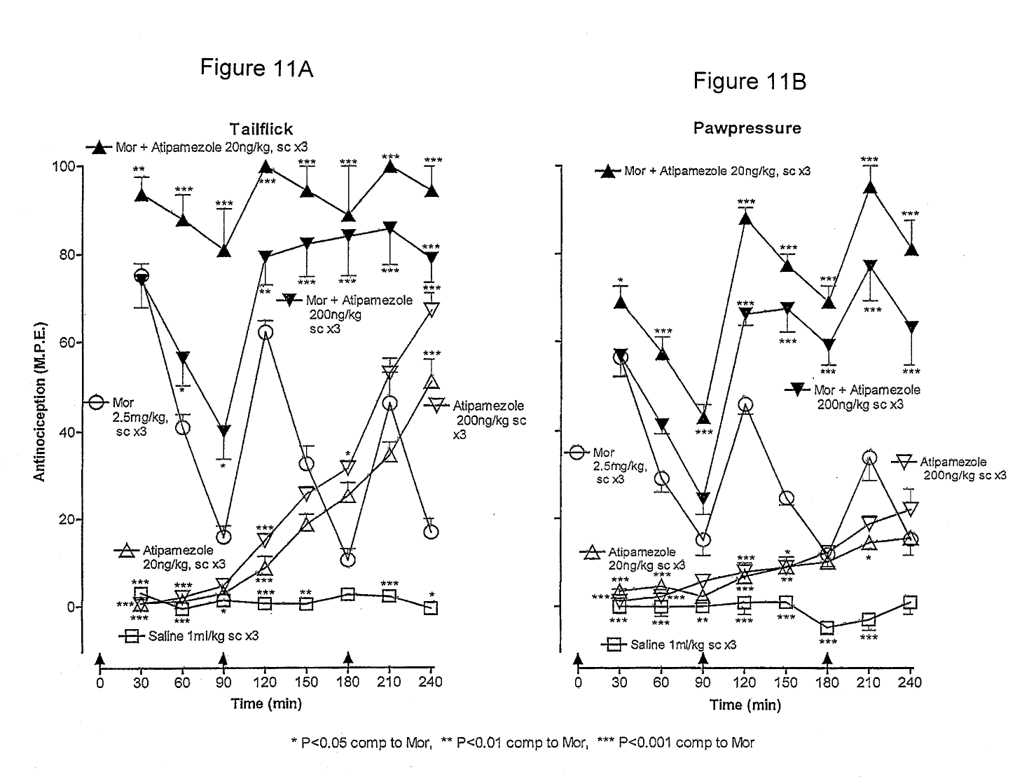

Additional experiments demonstrating the analgesic action of ultra-low doses of the non-selective alpha-2 receptor antagonist atipemazole were performed. Results are depicted in FIGS. 11A and 11B. Atipemazole was administered subcutaneously at a dose that is significantly lower than the alpha-2 receptor antagonist doses reported in the literature (see, e.g., Sabba et al. Anesthesiology 1994 80:1057-1072). In these experiments, the effects of the alpha-2 receptor antagonist atipemazole administered subcutaneously (sc) at a dose ineffective at causing alpha-2 receptor blockade on acute tolerance to the analgesic actions of spinal morphine in the tail flick test (FIG. 11A) and paw pressure test (FIG. 11B) in rats were examined.

Acute tolerance was produced by delivering three intrathecal successive injections of morphine (15 μg) at 90 minute intervals. Rats received morphine (2.5 mg/kg sc) alone, a combination of morphine (2.5 mg/kg sc) and a dose of atipemazole (20 ng/kg sc), a combination of morphine (2.5 mg/kg sc) and a dose of atipemazole (200 ng/kg sc), atipemazole alone (20 ng/kg sc), atipemazole alone (200 ng/kg, sc) or saline (1 ml/kg sc) at 90 minute intervals. The three repeated injections of morphine resulted in a rapid decay of the analgesic response in both tests. Combination of atipemazole with morphine significantly reduced the loss of response to morphine alone and sustained it near maximal level. In the tail flick test both doses of atipemazole alone produced a delayed analgesic response. A similar effect was elicited in the paw pressure test.

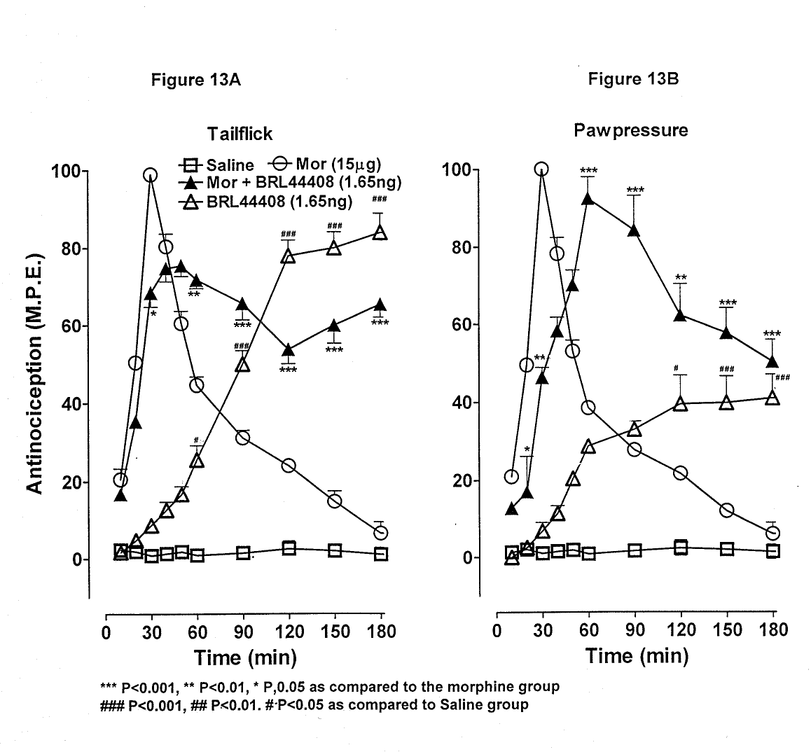

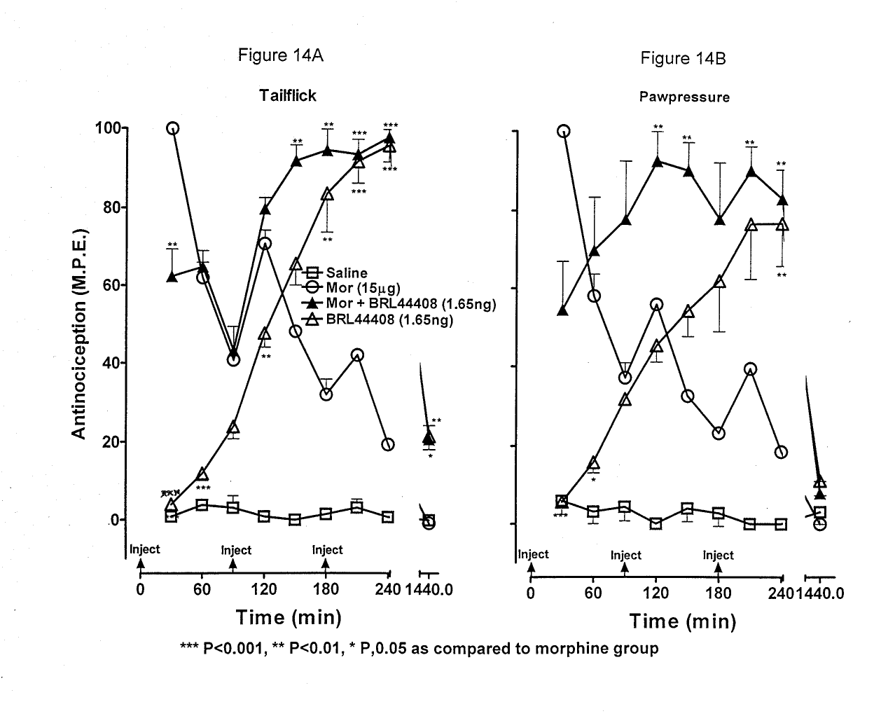

The effects of administration of an ultra-low dose of the selective alpha-2A receptor antagonist BRL 44408 at alleviating or delaying onset of pain were also examined. Results from these experiments are depicted in FIGS. 13A and 13B, and 14A and 14B. In these experiments, BRL 44408 was administered intrathecally at a dose ineffective at causing alpha-2A receptor blockade. The effects of BRL 44408 in augmenting morphine analgesia, following a single administration of morphine, are shown in FIGS. 13A (tail flick test) and 13B (paw pressure test). As shown, BRL 44408 administered once (1.65 ng) with morphine (15 μg) significantly increased morphine analgesia, relative to morphine alone (15 μg). The effects of BRL 44408 on acute tolerance to the analgesic actions of spinal morphine in the tail flick test (FIG. 14A) and paw pressure test (FIG. 14B) in rats were examined. In this study, acute tolerance was produced by delivering three intrathecal successive injections of morphine at 90 minute intervals. Rats were administered intrathecally morphine alone, a combination of morphine and BRL 44408 (1.65 ng), BRL 44408 alone (1.65 ng) or normal saline (20 μl) at 90 minute intervals. At a dose significantly below that producing the alpha-2A receptor blockage, BRL 44408 increased the acute analgesic action of morphine.

The effect of intrathecal administration of a low dose of BRL 44408 alone (1.65 ng) was also examined in rats. As shown in FIGS. 13A (tail flick test) and 13B (paw pressure test), a single administration of BRL 44408 produced significant analgesia, relative to saline administration, after 60 minutes. The analgesia increased and persisted for the duration of the study (180 minutes). As shown in FIGS. 14A (tail flick test) and 14B (paw pressure test), three successive administrations of BRL 44408, at 0, 90, and 180 minutes, produced similar analgesia.

In a further experiment, atipemazole (200 ng/kg, sc) administered to rats on a reverse light/dark cycle (wherein atipemazole was administered during the dark phase) did not produce a significant antinociceptive effect in the tail flick test. Baseline latencies were obtained every 5 minutes for 15 minutes prior to injection of either vehicle (saline 0.9%) or atipemazole and tested every 30 minutes for 3 hours. Data were converted to % maximum possible effect.

One explanation for the discrepancy from the above previous findings is testing of the animals during the dark phase of the light dark cycle (in the previous findings, animals were tested during the light phase). This was done because rats are nocturnal and more active for testing during their night. One known difference between nociceptive testing between light and dark phases is that morphine is 10 fold more potent in the dark phase compared to the light phase. For example, the ED50 dose for morphine during the light phase is 5 mg/kg, but is only 0.25 mg/kg during the dark phase.

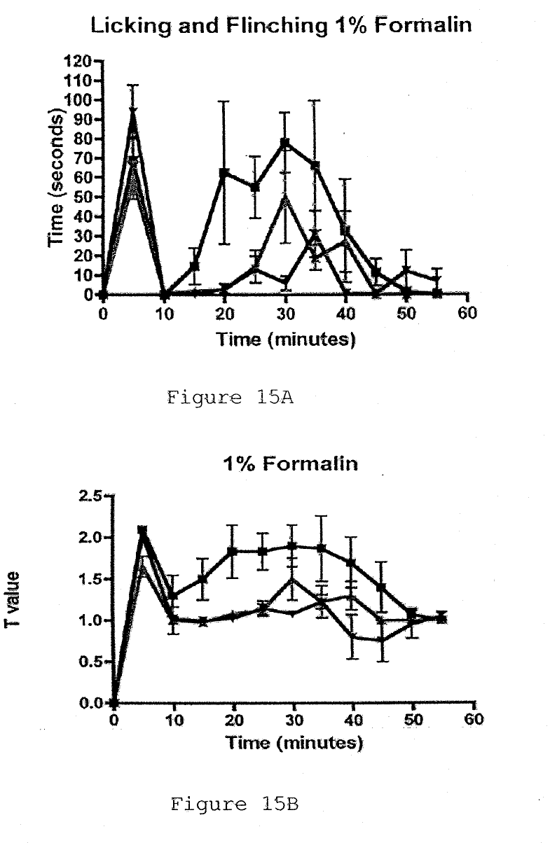

In a further experiment, the analgesic effect of atipemazole was examined using the formalin test, a persistent inflammatory pain model. The experiment was conducted with rats during the dark phase of the light-dark cycle. Rats were administered 1% formalin and pain behaviour was assessed by a weighted score. Atipemazole, morphine, or vehicle was injected by subcutaneous injection 20 minutes prior to formalin injection into the plantar surface of the rat hind paw. The behavior was evaluated in 5 min intervals, and the severity of the response was determined. As shown in FIGS. 15A and 15B, atipemazole alone was as effective as morphine in producing analgesia.

As will be understood by the skilled artisan upon reading this disclosure, embodiments of the invention should be construed and understood to include any alpha-2 receptor antagonist. Based on the teachings set forth in extensive detail elsewhere herein, the skilled artisan will understand how to identify such alpha-2 receptor antagonists, and combinations thereof, as well as the concentrations of alpha-2 receptor antagonists to use in such embodiments.

As demonstrated herein, alpha-2 receptor antagonists maybe administered, for example, epidurally, intrathecally, and systemically (e.g., orally, parenterally, subcutaneously, intramuscularly), where appropriate. Accordingly, the therapies described herein may be administered systemically or locally, and by any suitable route such as oral, buccal, sublingual, transdermal, subcutaneous, intraocular, intravenous, intramuscular or intraperitoneal administration, and the like (e.g., by injection) or via inhalation.

As used herein, the term “therapeutic compound” is meant to refer to an alpha-2 receptor antagonist.

As used herein “pharmaceutically acceptable vehicle” includes any and all solvents, excipients, dispersion media, coatings, antibacterial and antifungal agents, isotonic and absorption delaying agents, and the like which are compatible with the activity of the therapeutic compound and are physiologically acceptable to a subject. An example of a pharmaceutically acceptable vehicle is buffered normal saline (0.15 M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

Carrier or substituent moieties useful in the present invention may also include moieties which allow the therapeutic compound to be selectively delivered to a target organ. For example, delivery of the therapeutic compound to the brain may be enhanced by a carrier moiety using either active or passive transport (a “targeting moiety”). Illustratively, the carrier molecule may be a redox moiety, as described in, for example, U.S. Pat. Nos. 4,540,654 and 5,389,623, both to Bodor. These patents disclose drugs linked to dihydropyridine moieties which can enter the brain, where they are oxidized to a charged pyridinium species which is trapped in the brain. Thus drugs linked to these moieties accumulate in the brain. Other carrier moieties include compounds, such as amino acids or thyroxine, which can be passively or actively transported in vivo. Such a carrier moiety can be metabolically removed in vivo, or can remain intact as part of an active compound.

Structural mimics of amino acids (and other actively transported moieties) including peptidomimetics, are also useful in the invention. As used herein, the term “peptidomimetic” is intended to include peptide analogues which serve as appropriate substitutes for peptides in interactions with, for example, receptors and enzymes. The peptidomimetic must possess not only affinity, but also efficacy and substrate function. That is, a peptidomimetic exhibits functions of a peptide, without restriction of structure to amino acid constituents. Peptidomimetics, methods for their preparation and use are described in Morgan et al. (1989) (“Approaches to the discovery of non-peptide ligands for peptide receptors and peptidases,” In Annual Reports in Medicinal Chemistry (Virick, F. J., ed.), Academic Press, San Diego, Calif., pp. 243-253), the contents of which are incorporated herein by reference. Many targeting moieties are known, and include, for example, asialoglycoproteins (see e.g., Wu, U.S. Pat. No. 5,166,320) and other ligands which are transported into cells via receptor-mediated endocytosis (see below for further examples of targeting moieties which may be covalently or non-covalently bound to a target molecule).

The term “subject” as used herein is intended to include living organisms in which pain to be treated can occur. Examples of subjects include mammals such as, but not limited to, humans, apes, monkeys, cows, sheep, goats, dogs, cats, mice, rats, and transgenic species thereof. As would be apparent to a person of skill in the art, the animal subjects employed in the working examples set forth below are reasonable models for human subjects with respect to the tissues and biochemical pathways in question, and consequently the methods, therapeutic compounds and pharmaceutical compositions directed to same. As evidenced by Mordenti (J. Pharm. Sci. 1986 75(11):1028-40) and similar articles, dosage forms for animals such as, for example, rats can be and are widely used directly to establish dosage levels in therapeutic applications in higher mammals, including humans. In particular, the biochemical cascade initiated by many physiological processes and conditions is generally accepted to be identical in mammalian species (see, e.g., Mattson and Scheff, Neurotrauma 1994 11(1):3-33; Higashi et al. Neuropathol. Appl. Neurobiol. 1995 21:480-483). In light of this, pharmacological agents that are efficacious in animal models such as those described herein are believed to be predictive of clinical efficacy in humans, after appropriate adjustment of dosage.

Depending on the route of administration, the therapeutic compound may be coated in a material to protect the compound from the action of acids, enzymes and other natural conditions which may inactivate the compound. Insofar as the invention provides a combination therapy in which two therapeutic compounds are administered, each of the two compounds may be administered by the same route or by a different route. Also, the compounds may be administered either at the same time (i.e., simultaneously) or each at different times. In some treatment regimes it may be beneficial to administer one of the compounds more or less frequently than the other.

The compounds of the invention can be formulated to ensure proper distribution in vivo. For example, the blood-brain barrier (BBB) excludes many highly hydrophilic compounds. To ensure that the therapeutic compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., Ranade, V. V. J. Clin. Pharmacol. 1989 29(8):685-94). Exemplary targeting moieties include folate and biotin (see, e.g., U.S. Pat. No. 5,416,016 to Low et al.); mannosides (Umezawa et al. Biochem. Biophys. Res. Commun. 1988 153(3):1038-44; antibodies (Bloeman et al. FEBS Lett. 1995 357:140; Owais et al. Antimicrob. Agents Chemother. 1995 39(1):180-4); and surfactant protein A receptor (Briscoe et al. Am. J. Physiol. 1995 268 (3 Pt 1):L374-80). In a preferred embodiment, the therapeutic compounds of the invention are formulated in liposomes; in a more preferred embodiment, the liposomes include a targeting moiety.

Delivery and in vivo distribution can also be affected by alteration of an anionic group of compounds of the invention. For example, anionic groups such as phosphonate or carboxylate can be esterified to provide compounds with desirable pharmacokinetic, pharmacodynamic, biodistributive, or other properties.

To administer a therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. Prog. Clin. Biol. Res. 1984 146:429-34).

The therapeutic compound may also be administered parenterally (e.g., intramuscularly, intravenously, intraperitoneally, intraspinally, intrathecally, or intracerebrally). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g., vegetable oil). The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In some cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compound is mixed with at least one inert, pharmaceutically acceptable excipient or diluent or assimilable edible carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compound in the compositions and preparations may, of course, be varied. The amount of the therapeutic compound in such therapeutically useful compositions is such that a suitable dosage will be obtained.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Therapeutic compounds can be administered in time-release or depot form, to obtain sustained release of the therapeutic compounds over time. The therapeutic compounds of the invention can also be administered transdermally (e.g., by providing the therapeutic compound, with a suitable carrier, in patch form).

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compounds according to the invention are administered at a therapeutically effective dosage sufficient to achieve the desired therapeutic effect of the opioid receptor agonist, e.g. to mitigate pain and/or to effect analgesia in a subject, to suppress coughs, to reduce and/or prevent diarrhea, to treat pulmonary edema or to alleviate addiction to opioid receptor agonists. For example, if the desired therapeutic effect is analgesia, the “therapeutically effective dosage” mitigates pain by about 25%, preferably by about 50%, even more preferably by about 75%, and still more preferably by about 100% relative to untreated subjects. Actual dosage levels of active ingredients in the pharmaceutical compositions of this invention may be varied so as to obtain an amount of the active compound(s) that is effective to achieve and maintain the desired therapeutic response for a particular subject, composition, and mode of administration. The selected dosage level will depend upon the activity of the particular compound, the route of administration, frequency of administration, the severity of the condition being treated, the condition and prior medical history of the subject being treated, the age, sex, weight and genetic profile of the subject, and the ability of the therapeutic compound to produce the desired therapeutic effect in the subject. Dosage regimens can be adjusted to provide the optimum therapeutic response. For example, several divided doses may be administered daily or the dose may be proportionally reduced as indicated by the exigencies of the therapeutic situation.

However, it is well known within the medical art to determine the proper dose for a particular patient by the dose titration method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed. Also, dosages are routinely determined through preclinical ADME toxicology studies and subsequent clinical trials as required by the FDA or equivalent agency. The ability of an opioid receptor agonist to produce the desired therapeutic effect may be demonstrated in various well known models for the various conditions treated with these therapeutic compounds. For example, mitigation of pain can be evaluated in model systems that may be predictive of efficacy in mitigating pain in human diseases and trauma, such as animal model systems known in the art (including, e.g., the models described herein).

Compounds of the invention may be formulated in such a way as to reduce the potential for abuse of the compound. For example, a compound may be combined with one or more other agents that prevent or complicate separation of the compound therefrom.

The contents of all cited publications are incorporated herein by reference in their entirety.

The following nonlimiting examples are provided to further illustrate the present invention.

### EXAMPLES

### Example 1

### Animals

Experiments were conducted using adult male Sprague-Dawley rats (Charles River, St. Constant, QC, Canada) weighing between 200-250 grams. Animals were housed individually in standard laboratory cages, maintained on a 12-hour light/dark cycle, and provided with food and water ad libitum. The surgical placement of chronic indwelling intrathecal catheters (polyethylene PE 10 tubing, 7.5 cm) into the spinal subarachnoid space was made under 4% halothane anesthesia, using the method of Yaksh and Rudy Physiol. Behav. 1976 7:1032-1036). Specifically, the anesthetized animal was placed prone in a stereotaxic frame, a small incision made at the back of the neck, and the atlanto-occipital membrane overlying the cisterna magna was exposed and punctured with a blunt needle. The catheter was inserted through the cisternal opening and slowly advanced caudally to position its tip at the lumbar enlargement. The rostral end of the catheter was exteriorized at the top of the head and the wound closed with sutures. Animals were allowed 3-4 days recovery from surgery and only those free from neurological deficits, such as the hindlimb or forelimb paralysis or gross motor dysfunction, were included in the study. All drugs were injected intrathecally as solutions dissolved in physiological saline (0.9%) through the exteriorized portion of the catheter at a volume of 10 μl followed by a 10 μl volume of 0.9% saline to flush the catheter.

### Example 2

### Assessment of Nociception

The response to brief nociceptive stimuli was tested using two tests: the tail flick test and the paw pressure test.

The tail flick test (D'amour & Smith, J. Pharmacol. Exp. Ther. 1941 72:74-79) was used to measure the response to a thermal nociceptive stimulus. Radiant heat was applied to the distal third of the animal's tail and the response latency for tail withdrawal from the source was recorded using an analgesia meter (Owen et al., J. Pharmacol. Methods 1981 6:33-37)). The stimulus intensity was adjusted to yield baseline response latencies between 2-3 seconds. To minimize tail damage, a cutoff of 10 seconds was used as an indicator of maximum antinociception.

The paw pressure test (Loomis et al., Pharm. Biochem. 1987 26:131-139) was used to measure the response to a mechanical nociceptive stimulus. Pressure was applied to the dorsal surface of the hind paw using an inverted air-filled syringe connected to a gauge and the value at which the animal withdrew its paw was recorded. A maximum cutoff pressure of 300 mmHg was used to avoid tissue damage. Previous experience has established that there is no significant interaction between the tail flick and paw pressure tests (Loomis et al., Can. J. Physiol. Pharmacol. 1985 63:656-662).

### Example 3

### Determination of Inhibition of Clonidine and/or Morphine Analgesia by Alpha-2 Receptor Antagonists

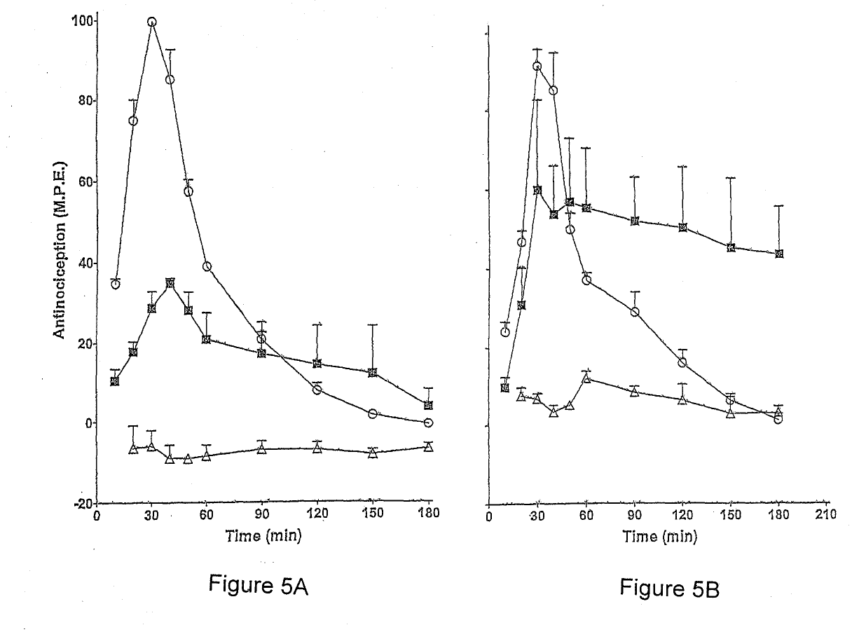

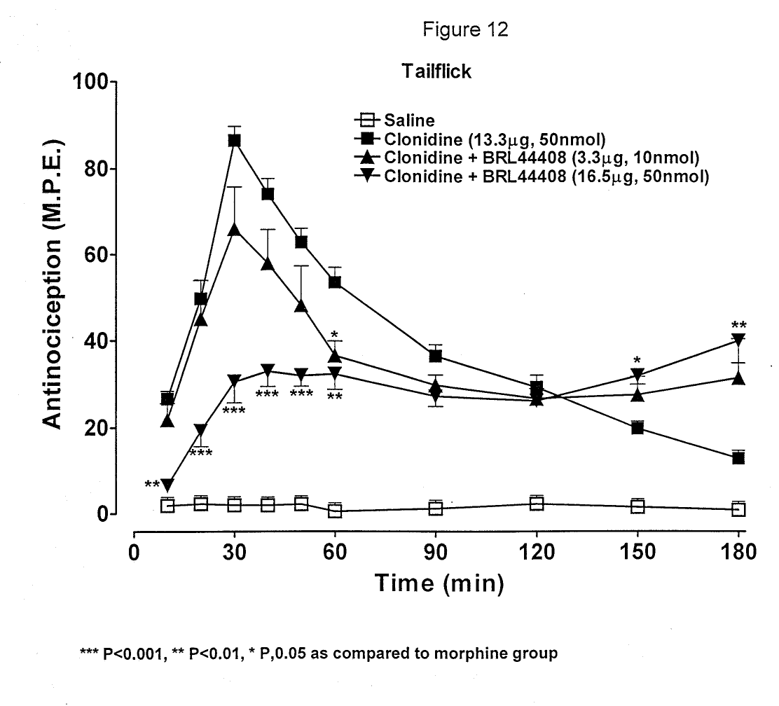

The effects of atipemazole, yohimbine, idazoxan and mirtazapine, as well as the selective alpha-2A receptor antagonist BRL 44408, were tested on the acute analgesic action of spinal clonidine to establish that each of these drugs act as alpha-2 receptor antagonists. A single injection of clonidine was administered intrathecally and the response measured in the tail flick and paw pressure test. In subsequent tests, clonidine was delivered in combination with 1, 5 or 10 μg atipemazole, 30 μg yohimbine, 10 μg idazoxan, 2 μg mirtazapine, or 3.3 μg or 16.5 μg of BRL 44408 (tail flick only). Following drug administration, nociceptive testing was performed every 10 minutes for the first 60 minutes and every 30 minutes for the following 120-150 minute period. Results for atipemazole are depicted in FIG. 1A (tail flick) and FIG. 1B (paw pressure). Results for yohimbine are depicted in FIG. 4A (tail flick) and FIG. 4B (paw pressure). Results for idazoxan are depicted in FIG. 9A (tail flick) and FIG. 9B (paw pressure). Results for mirtazapine are depicted in FIG. 7A (tail flick) and FIG. 7B (paw pressure). Similar experiments were performed with yohimbine at 30 μg in combination with morphine. See FIG. 5A (tail flick) and FIG. 5B (paw pressure). Results for BRL 44408 are shown in FIG. 12, where an antagonistic dose (16.5 μg) antagonized clonidine analgesia. The 3.3 μg dose did not antagonize clonidine, and thus a dose about 1000 fold lower (1.65 ng) was used for the studies with morphine or BRL 44408 alone.

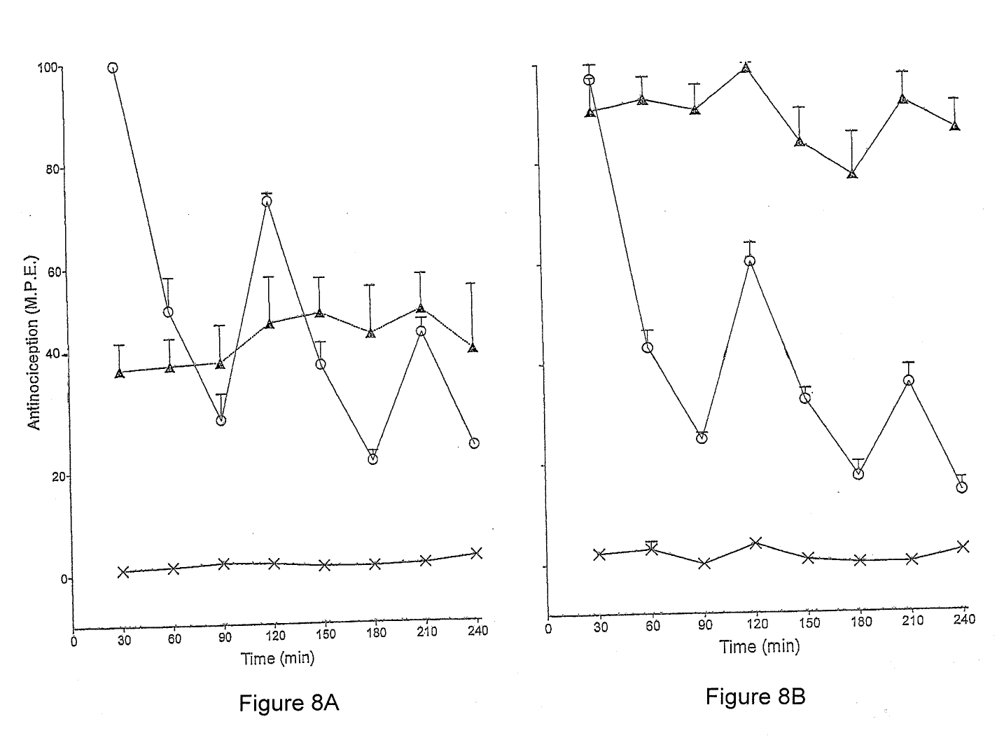

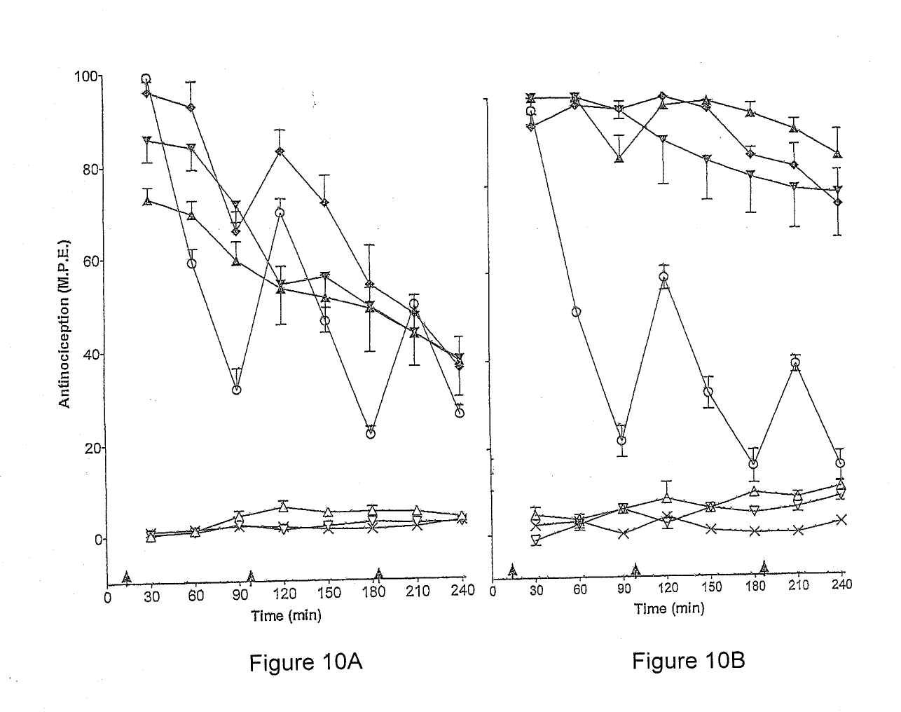

The effects of ultra-low doses of atipemazole, yohimbine, idazoxan and mirtazapine, as well as the selective alpha-2A receptor antagonist BRL 44408, were also tested for potentiation of acute analgesic action of spinal or subcutaneous morphine. Acute opioid tolerance was induced by three serial intrathecal morphine injections (15 μg) or systemic morphine injections (2.5 mg/kg sc). Rats were administered morphine alone, a combination of morphine (15 μg intrathecally or 2.5 mg/kg sc) and an ultra low dose of atipemazole, yohimbine, idazoxan, mirtazapine, or BRL 44408. Results for atipemazole are depicted in FIGS. 2A, 2B, 11A and 11B. Results for yohimbine are depicted in FIGS. 6A and 6B. Results for idazoxan are depicted in FIGS. 10A and 10B. Results for mirtazapine are depicted in FIGS. 8A and 8B. Results for BRL 44408 are depicted in FIGS. 14A and 14B.

The analgesic effect of an ultra-low dose of an alpha-2 receptor antagonist administered alone was examined. Atipemazole was administered systemically, in three successive administrations, at 20 ng/kg or 200 ng/kg. The results are shown in FIGS. 11A and 11B.

The analgesic effect of an ultra-low dose of an alpha-2A receptor antagonist administered alone was also examined. BRL 44408 was administered intrathecally, in a single administration, at 1.65 ng. The results are shown in FIGS. 13A and 13B. In another study, three successive doses of BRL 44408 (1.65 ng) were administered intrathecally. The results are shown in FIGS. 14A and 14B.

### Example 4

### Data Analysis

For the in vivo studies, tail flick and paw pressure values were converted to a maximum percentage effect (M.P.E.): M.P.E.=100×[post-drug response−baseline response]/[maximum response−baseline response]. Data represented in the figures are expressed as mean (±S.E.M.). The ED₅₀ values were determined using a non-linear regression analysis (Prism 2, GraphPad Software Inc., San Diego, Calif., USA). Statistical significance (p<0.05, 0.01. or 0.001) was determined using a one-way analysis of variance followed by a Student Newman-Keuls post hoc test for multiple comparisons between groups.

### Example 5

### Analgesic Effect of Atipemazole in a Persistent Inflammatory Pain Model

The formalin test is a widely used tonic model of continuous pain resulting from formalin-induced tissue injury. It is a useful model, particularly for the screening of novel compounds, since it encompasses inflammatory, neurogenic, and central mechanisms of nociception. We used 1% formalin and assessed pain behavior by a weighted score. Drugs or vehicle were injected by subcutaneous injection 20 minutes prior to formalin injection into the plantar surface of the rat hind paw.

Briefly, the nociceptive behavior was assessed as follows: (1) no favouring of the injected hind paw, (2) favouring, (3) complete elevation of the hind paw from the floor, and (4) licking or flinching. The behavior was evaluated in 5 min intervals, and the severity of the response was determined by the following formula: (0×the time spent in category 1, 1×the time spent in category 2, 2×the time spent in category 3, 3×the time spent in category 4).

The data (FIGS. 15A and 15B) show that atipemazole (alone) was as effective as morphine. Data represent means+/−s.e.m. for N=4.

What is claimed is:

1. A method for alleviating or delaying onset of pain in a subject comprising systemically administering to the subject an ultra low dose of an alpha-2 receptor antagonist which does not significantly reduce, block, or inhibit alpha-2 receptor activity.

2. The method of claim 1 wherein the alpha-2 receptor antagonist is selected from the group consisting of atipemazole (or atipamezol or atipamzole), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563, A 80246 mesylate, ARC 239 dihydrochloride, imiloxan hydrochloride, JP 1302 dihydrochloride, rauwolscine hydrochloride, RS 79948, spiroxatrine, and yohimbine.

3. The method of claim 1 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, mianserin, and tolazoline.

4. The method of claim 1 wherein the alpha-2 receptor antagonist is a selective alpha-2A receptor antagonist.

5. The method of claim 4 wherein the selective alpha-2A receptor antagonist is BRL 44408 or BRL 48962.

6. The method of claim 5, wherein the selective alpha-2A receptor antagonist is BRL 44408.

7. The method of claim 6, wherein the BRL 44408 is administered subcutaneously.

8. The method of claim 1, wherein the alpha-2 receptor antagonist is administered subcutaneously.

9. The method of claim 1, wherein the alpha-2 receptor antagonist is atipemazole.

10. The method of claim 9, wherein the atipemazole is administered subcutaneously.

11. A method for alleviating or delaying onset of pain in a subject comprising administering to the subject an ultra low dose of an alpha-2 receptor antagonist which does not significantly reduce, block, or inhibit alpha-2 receptor activity;
wherein the alpha-2 receptor antagonist is BRL 44408.

12. The method of claim 11, wherein BRL 44408 is administered intrathecally.
